(19) United States
(12) Patent Application Publication
(10) Pub. No.: US 2024/0359176 A1
(43) Pub. Date: Oct. 31, 2024

(54) DISPOSABLE FLUIDIC CARTRIDGE FOR INTERFEROMETRIC REFLECTANCE IMAGING SENSOR

(71) Applicant: TRUSTEES OF BOSTON UNIVERSITY, Boston, MA (US)

(72) Inventors: M. Selim ÜNLÜ, Newton Highlands, MA (US); Derin SEVENLER, Brookline, MA (US); Jacob TRUEB, Arlington, MA (US); Steven SCHERR, Arlington, MA (US)

(73) Assignee: TRUSTEES OF BOSTON UNIVERSITY, Boston, MA (US)

(21) Appl. No.: 18/657,525

(22) Filed: May 7, 2024

Related U.S. Application Data
(63) Continuation of application No. 16/772,004, filed on Jun. 11, 2020, now abandoned.
(60) Provisional application No. 62/597,544, filed on Dec. 12, 2017.

(51) Int. Cl. B01L 3/00; B01L 9/00; G01N 15/1434

(52) U.S. Cl. CPC B01L 3/502715; B01L 9/527; G01N 15/1434; B01L 2200/025; B01L 2200/027; B01L 2200/04; B01L 2200/12; B01L 2300/041; B01L 2300/12; B01L 2300/168; G01N 2015/1454

(57) ABSTRACT

A low cost/disposable fluidic cartridge for interferometric reflectance imaging sensor is described. Systems and methods using this cartridge are also disclosed. The cartridges and systems simplify the protocols and minimizes potential user error, for example, in biosensing experiments and assays.

### CROSS-REFERENCE TO RELATED APPLICATIONS

This application is a continuation under 35 U.S.C. § 120 of co-pending U.S. application Ser. No. 16/772,004, filed Jun. 11, 2020, which is a 35 U.S.C. § 371 National Phase Entry of International Application No. PCT/US2018/064927 filed Dec. 22, 2018, which designates the US., and which claims any and all benefits as provided by law including benefit under 35 U.S.C. § 119(e) of the U.S. Provisional Application No. 62/597,544, filed Dec. 12, 2017, the contents of which are incorporated herein by reference in their entireties.

### FIELD OF THE INVENTION

This invention relates to systems and methods for measuring single particles using optical methods. More specifically, to disposable fluidic cartridges for interferometric measurements.

### BACKGROUND

Microfluidic systems allow biomarker detection experiments to be conducted in an enclosed system under controlled conditions. These systems simplify the protocols and minimize potential user error. Another advantage of incorporating microfluidics is that it can increase mass transport of the kinetic species and allows the efficient use of smaller sample volumes.

In some systems, there can be specific requirements for cartridge designs for different biosensing modalities. Some microfluidic cartridge platforms can be manufactured using low-cost cyclo-olefin copolymer (COC) films, (Scherr S M, Daaboul G G, Trueb J, Sevenler D, Fawcett H, Goldberg B, Connor J H, Ünlü MS, “Real-time capture and visualization of individual viruses in complex media,”ACS Nano 2016; 10(2): 2827-33; S. M. Scherr, D. S. Freedman, K. N. Agans, A. Rosca, E. Carter, M. Kuroda, H. Fawcett, C. Mire, T. W. Geisbert, M. S. Ünlü, and J. H. Connor, “Disposable cartridge platform for rapid detection of viral hemorrhagic fever viruses,”Lab Chip, Vol. No. 10., February 2017). However, such materials are difficult to machine or otherwise fabricate to allow for flat viewing windows along with ports for fluid input and output. Often times such fabrication methods result in expensive cartridges.

There is therefore a need for a sensing cartridge that is inexpensive to manufacture and is easy to use while also having suitable optical characteristics. In addition, controlling the cost would allow for disposable cartridges to be manufacture which would be convenient and have practical advantages, for example, in use as part of an advanced diagnostic tool for pathogen detection or as part of a biological nanoparticle visualization and characterization tool or as part of a molecular kinetics measurement tool.

### SUMMARY

The technology described herein relates to low-cost and disposable cartridges, which are useful and highly desirable for biosensing and molecular diagnostics. It has been found that, for optical biosensors, especially those that require imaging of the sensor surface, the requirements are very specific and restrict the use of common materials such as plastics for the cartridge construction. For example, use of a plastic optical window is detrimental to Single Particle-Interferometric Reflectance Imaging Sensor (SP-IRIS) measurements, in particular in the polarization enhanced modality. Therefore, the requirements for optical imaging necessitate the top window to be of optical quality and typical injection molded plastics may not be used. It is desirable to have a glass (or other high optical quality material) viewing window. However, such materials are difficult to machine or otherwise fabricate to allow for flat viewing windows along with ports for fluid input and output. Often times such fabrication methods result in expensive cartridges. Furthermore, fluidic connections through the top of the cartridge often interfere with the imaging optics, such as microscope objectives, necessitating larger cartridge dimensions to provide the required separation between fluidic connectors. On the other hand, routing the fluidic connection to the bottom of the cartridge would require more complex 3-D channel geometry.

In one aspect the invention includes an optical biosensing cartridge comprising: a substrate with through holes as ports to facilitate liquid flow, a cover window having a transparent portion, a spacer separating the cover window from the substrate by a predefined distance, a channel extending from one port to a different port, the channel defined by the substrate, spacer and cover film, and a detection region on the substrate at least partially in the channel, wherein the detection region includes at least one dielectric layer having a predefined uniform thickness. In some embodiments the substrate comprises silicon. In some embodiments the detection region includes a layer of silicon oxide on a silicon substrate. In some embodiment the detection region includes a layer of silicon nitride on a silicon substrate. In some embodiment the through holes are formed by laser micromachining. In some embodiment the through holes include one through hole for liquid inlet and one through hole for liquid outlet. In some embodiments the spacer comprises an adhesive. In some embodiment the cover window is glass and includes an anti-reflection coating on a top surface. In some embodiments the cover window comprises an optical grade transparent material such as quartz or borosilicate glass. In some embodiments the detection region includes one or more of identification regions, alignment marks for robotic spotting, reflective reference regions and autofocus regions. In some embodiments the substrate is a silicon chip, and the ports comprise a first port and the second port is configured as through the Si chip holes manufactured by laser micromachining, the spacer includes a pressure sensitive adhesive, the detection area includes a dielectric layer of silicon oxide or silicon nitride between 50-200 nm thick, and the cover window comprises silicate glass having one surface comprising an anti-reflective coating.

In another aspect the invention includes an apparatus comprising an optical biosensing cartridge as described herein and a holder, wherein the holes of the cartridge include a first port and a second port and the channel extends between the first port and the second port, and wherein the holder includes, a base having a first fluid conduit coupled to a first port, and a second fluid conduit coupled to a second port, and, a clamping element for removably fastening the cartridge to the base; wherein the first conduit, the first port, the channel, the second port and the second conduit define a fluid flow path from the first fluid conduit to the second fluid conduit.

In another aspect, the invention includes an apparatus comprising a removable cartridge and holder. The cartridge includes, a substrate having a first port and a second port, a cover film having a transparent portion, a spacer separating the cover film from the substrate by a predefined distance, a channel extending from the first port to the second port, the channeled defined by the substrate, spacer and cover film, and a detection region on the substrate at least partially in the channel, wherein the detection region includes one or multiple dielectric layers having a predefined uniform thickness. The holder of the apparatus includes a base having a first fluid conduit coupled to the first port, and a second fluid conduit coupled to the second port. The holder of the apparatus also includes a clamping element for removably fastening the cartridge to the base. In some embodiments of the apparatus, the first conduit, the first port, the channel, the second port and the second conduit define a fluid flow path from the first fluid conduit to the second fluid conduit. In some embodiments, the base includes an alignment element including at least three pins positioned to alignment the cartridge with the base whereby the first fluid conduit is aligned with first port of the cartridge and the second fluid conduit is aligned with the second port of the cartridge. In some embodiments the transparent portion comprises an optical grade transparent material such as quartz or borosilicate glass. In some embodiments the transparent portion is aligned with the detection region. In some embodiments the detection region includes one or more of identification regions, alignment marks for robotic spotting, reflective reference regions and autofocus regions. In some embodiments of the apparatus, the clamping element includes: a platform supporting the base, a rotary cam mounted to the platform and engaging a bottom platform through a cam follower mounted on the bottom platform, wherein the rotary cam has a loading position and a clamped position, a clamping bar for engaging the cartridge and compressing the cartridge against the base, the clamping bar having an aperture in alignment with at least a portion of the transparent portion, at least one guide rail mechanically coupled to the platform and bottom platform and for holding the platform and clamping bar in alignment during operation of the rotary cam during a clamping operation, and at least one spring mechanically coupled to the base, bottom platform, clamping bar and the cam follower; wherein when the rotary cam is in the clamped position the bar provides a compressive force through the spring against the cartridge, and when the rotary cam is in a loading position the cartridge can be disengaged and removed from the base. In some embodiments the apparatus further comprises a first sealing element disposed on the base at one end of the first fluid conduit for sealing a connection between the first fluid conduit and the first port, and a second sealing element disposed on the base at one end of the second fluid conduit for sealing a connection between the second fluid conduit and the second port. In some embodiments the rotary cam includes a handle and the platform includes a first bumper and a second bumper for engaging the handle and restricting the rotational motion of the cam to the clamped and unclamped position.

In yet another aspect, the invention includes a system for measuring particles comprising an apparatus as herein described, an Interferometric Reflectance Imaging Sensor (IRIS) system comprising an objective lens for illuminating the detection region of the cartridge and collecting reflected light from the detection region, and a stage for holding the apparatus of claim 1 and for moving the apparatus relative to the objective lens. In some embodiments the substrate is a first reflective surface, the dielectric layer comprises a second reflective surface, and single particles are detected on the dielectric surface by the IRIS system.

In another aspect the invention includes a method of measuring particles or a biomass accumulated on a sensor surface, comprising flowing an analyte solution including particles through the channel and the detection region of an apparatus as described herein and measuring the particles in the detection region using a system as described herein.

Therefore, embodiments described herein provided cartridges having optical imaging viewing windows such a made of glass (or other high optical quality material). These cartridges can be easily and economically machined or otherwise fabricated and allow, along with flat high quality optical windows, ports for fluid input and output to be easily made, and obviates any need for more complex 3-D channel geometry. Furthermore, the apparatus described herein allow for quick and easy fluidic connections to the cartridge that do not interfere with the imaging optics, such as microscope objectives, in imaging systems.

These and other capabilities of the invention, along with the invention itself, will be more fully understood after a review of the following figures, detailed description, and claims.

### BRIEF DESCRIPTION OF THE FIGURES

The accompanying drawings, which are incorporated into this specification, illustrate one or more exemplary embodiments of the inventions and, together with the detailed description, serve to explain the principles and applications of these inventions. The drawings and detailed description are illustrative, and are intended to facilitate an understanding of the inventions and their application without limiting the scope of the invention. The illustrative embodiments can be modified and adapted without departing from the spirit and scope of the inventions. The patent or application file contains at least one drawing executed in color. Copies of this patent or patent application publication with color drawings will be provided by the Office upon request and payment of the necessary fee.

FIG. 1 Illustrates an embodiment of a configuration for a sensor chip.

FIG. 2 shows an embodiment of an IRIS sensor chip.

FIG. 3 shows a schematic drawing of a microfluidic cartridge.

FIG. 4 Panels 1-5 illustrate steps in a protocol for a detection experiment. Panel 1 shows an analyte labeling step. Panel 2 shows an addition step to a flow cell. Panel 3 shows a cap sealing step. Panel 4 is a detailed view of showing flow of the analyte mixture. Panel 5 shows removal of an adhesive seal from a flow cell reservoir.

FIG. 5A shows a blown up view of an embodiment of a cartridge. FIG. 5B shows a front cross cut view of the cartridge in a clamping apparatus. FIG. 5C shows another blown up view of the embodiment of a cartridge. FIG. 5D is a picture of a components of a cartridge according to some embodiments of the invention.

FIG. 6A is a top view of a substrate for a cartridge according to some embodiments. FIG. 6B is a detailed view showing a chip identification region. FIG. 6C is a detailed view showing a reference region. FIG. 6D is detailed view showing an auto focus region. FIG. 6E is a picture of patterned six-inch wafer according to some embodiments for making a cartridge.

FIG. 7A is an exploded view showing an embodiment of a fluid manifold and a cartridge. FIG. 7B shows a front cross section view of the holder and cartridge.

FIG. 8A shows a cross section view of a holder for cartridges according to some embodiments. FIG. 8B is a detail view of some of the components of the holder while FIG. 8C shows details of some components is an exploded view.

FIG. 9A is an isometric view of a holder in a clamped position according to some embodiments. FIG. 9B shows a cross-cut side view of the holder in the clamped position. FIG. 9C shows an isometric view of the holder in a loading position. FIG. 9D show a cross-cut side view of the holder in the loading position.

### DETAILED DESCRIPTION

The present invention is directed to a disposable cartridge that is suitable for use for biosensors utilizing optical imaging of the sensor surface on which biomolecules, and nanoscale biological particles are captured. For example, the cartridge can be used with Interferometric Reflectance Imaging Sensor (IRIS) or single-particle-IRIS (SP-IRIS). In addition, apparatus for holding and aligning the cartridge including fluid connection are described. Systems including the cartridge, a holder and IRIS are also provided.

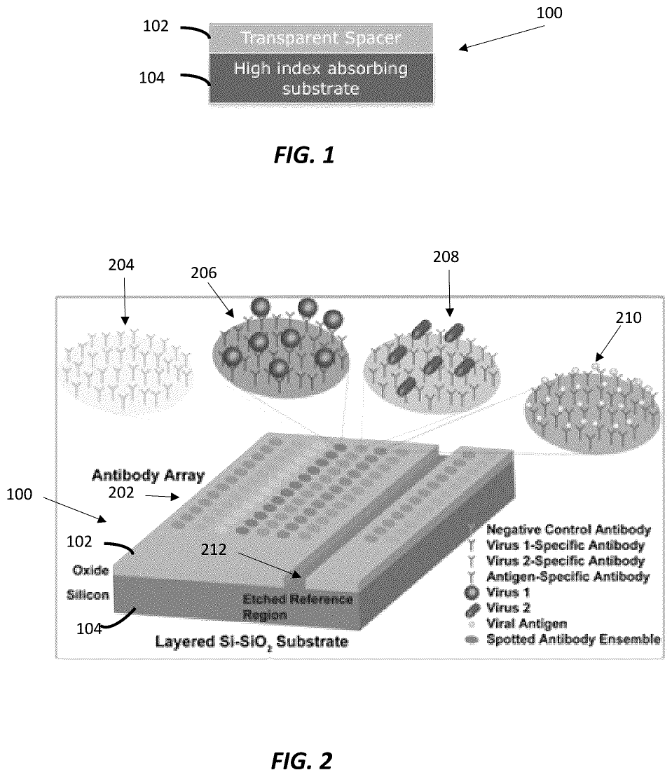

IRIS is a biosensor modality that requires high quality optical images acquired as analytes bind to the sensor surface. These images are acquired through the top window. The sensor chip itself has specific requirements including (i) flat and smooth surface that can be chemically functionalized and (ii) a multi-layer (at least two dielectric layers) structure to facilitate interference signature and (iii) desirably an absorbing substrate to eliminate any stray light. It is also desirable that the selection of materials for the sensor chips allow for scalable manufacturing. A basic configuration of the sensor chip is shown in FIG. 1. The substrate 100 includes a top transparent spacer 102 provides the spectral signature for Interferometric Reflectance Imaging Sensor (IRIS). The top layer has a relatively low refractive index to achieve high sensitivity sensing of binding of biological particles and molecules on the surface. Due to the low refractive index of common biological molecules and particles, for example refractive index for viruses and proteins is around 1.5, for exosomes is around 1.4, it is desirable to have the top layer of the IRIS sensor to have a comparable refractive index. The bottom part of the substrate 104 can have a higher refractive index providing the reflection of the reference optical field and it is desirable to have an absorbing material to eliminate stray light. One nearly ideal configuration for sensors operating with visible light is silicon oxide or silicon nitride coated Si substrates. Typical silicon oxide and silicon nitride, and their combinations provide refractive indices in the range of 1.45 to 2.3 in the visible spectrum of light.

As illustrated in FIG. 2, in some embodiments, a top surface of silicon oxide 102 (or silicon nitride) can be functionalized readily and capture probes for multiplexed detection of analytes can be arrayed on the surface. Some features shown include an antibody array 202, over the layered silicon/silicon oxide substrate 100 on the transparent layer 102 where the array can include negative control antibodies 204, a first virus specific antibody 206 shown with a first virus, a second virus specific antibody 208 shown with a second virus, and an antigen specific antibody 210 shown with a viral antigen. An etched reference region 212 is also shown.

In some embodiments for the incorporation of the IRIS chip in a fluidic system, the cartridge is self-contained, manufacturable, is of sufficient optical quality, is non-fouling, utilizes room temperature bonding for microarray chip addition, and fits beneath an imaging objective, all while maintaining the sensitivity of the assay.

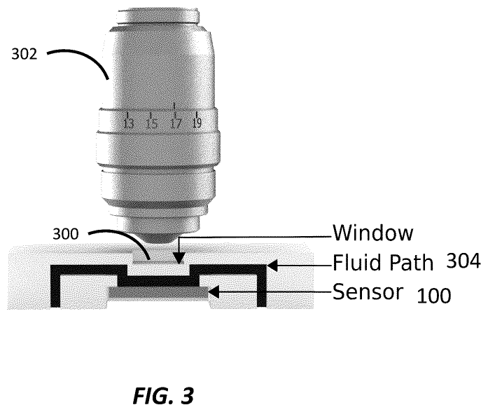

FIG. 3 shows a schematic drawing of a microfluidic cartridge illustrating how the sensor chip is incorporated in the incubation chamber and sensor surface is imaged through a transparent window. The schematic configuration shows liquid flow managed through a 3-D cartridge design that can be fabricated by multi-layer laminates or bonding together injection molded plastics. The substrate 100 and window 300 are shown as well as an optical imaging apparatus component 302 and a fluid flow path 304. This is disclosed in commonly own International Application (designating the U.S.) nos. PCT/US2010/033397, PCT/US2014/062605 and PCT/US2015/019136, which are hereby incorporated by reference, in their entirety. In some embodiments, the sensor is a solid sensor chip that can be fabricated by standard Si processing techniques.

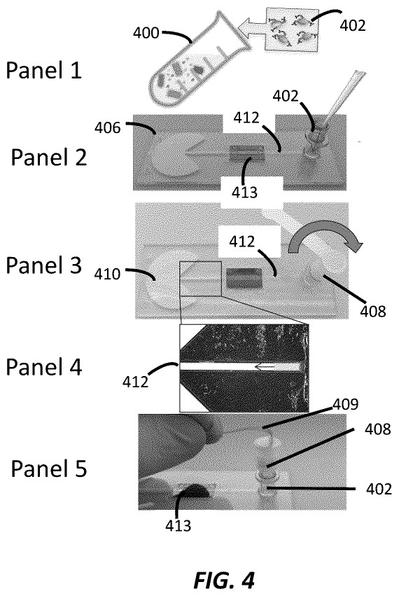

A protocol for detection experiments is illustrated in FIG. 4 as a series of panels 1-5. For direct particle detection, such as virus detection, the steps depicted by panels 2-5 are used. For detection of an analyte not directly detectable, such as a protein, the step depicted as panel 1 is added wherein the analyte of interest is decorated with a suitable particle.

In panel 1, as exemplified for protein assays in FIG. 4, a sample 400 containing the protein and a nano-particle, such as a gold nanoparticle (AuNP), labeled antibody (Ab) are combined to form a Protein-Ab-NP conjugate 402. In this manner, individual proteins (e.g., with Au-NP tags) have sufficient optical signature to be detected as they bind to the sensor surface. In some embodiments these NP are small, such as between 1 and 100 nm, adverse mass transport limitations can be avoided. In some embodiment nanoparticle including metal (e.g., gold, silver, pallidum, platinum, copper), metal compounds such as oxides, chalcogenides, chlorides, metalorganic compounds, as organic compounds and polymers (e.g., resin beads), and reaction products of these (e.g., a resin including metal decoration) can be used.

In panel 2, an aliquot, e.g., 100 μL, of the sample is transferred to the bottom of a reservoir 404 which is in fluid communication with a flow cell 406. A cap 408 is used to seal the reservoir. In some embodiments a luer cap sealed with adhesive strip 409 that can be screwed down is used. The solution flows towards absorptive pad 410 through channel 412 and detection region 413.

Panel 4 is a detailed view showing the adsorptive pad and channel. When liquid, moving as indicated by the arrow, reaches the absorptive pad the cap is vented. For example, in embodiments using a luer cap sealed with adhesive strip, the adhesive strip is removed as shown by panel 5. The cartridge can then be placed in an instrument, such as SP-IRIS instrument to begin data acquisition. The detection region is shown as 413.

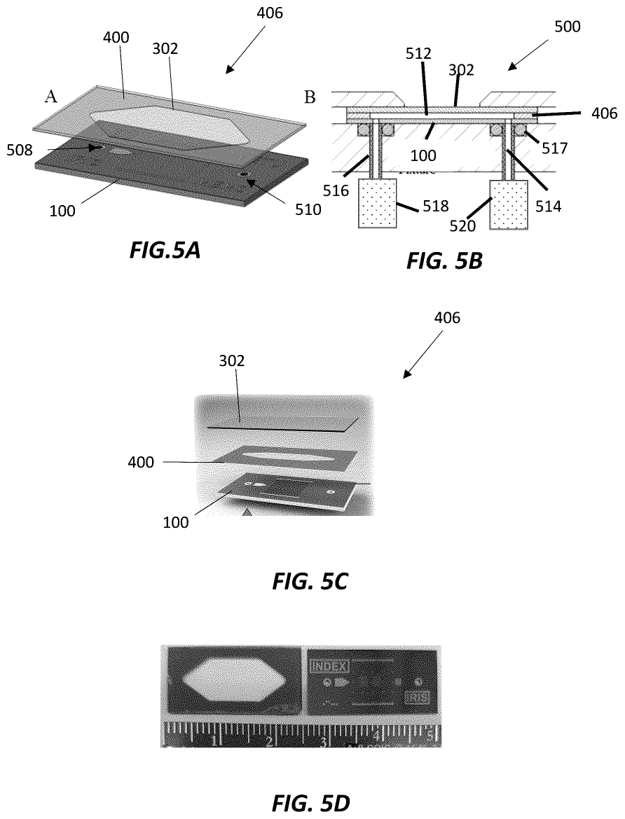

FIG. 5A shows a blown up view of an embodiment of a cartridge 406, while FIG. 5B shows a front cross cut view of the cartridge in a clamping apparatus 500. FIG. 5C shows another blown up view of the cartridge. The cartridge includes a substrate 100 which can be a silicon substrate. The cartridge also includes a cover window 302 including a transparent portion and a spacer 400. In some embodiments the window is a cover glass or any other flat window material. The substrate also includes ports 508 and 510 which are holes etched, cut, or drilled through Si substrate to facilitate liquid flow therethrough. The spacer of the cover window is configured to contact a substrate face so that the transparent portion and spacer define a channel 512 extending between the ports 508 and 510. The clamping apparatus can include fluid conduit 514 which can engage with the port 510, and fluid conduit 516 which can engage with port 508. The clamping apparatus can also include sealing elements 517 (e.g., O-rings). In some embodiments, the channels can be connected to a fluid source and sink, for example, a first reservoir 518 connected to a pump for pumping fluid into fluid conduit 516, through port 508, through channel 512, through port 510, through channel 514 and to a second reservoir 520. In some embodiments the first and second reservoir are connected, or are the same element, for example creating a loop of fluid flow. In some embodiments the fluid flow can be in the reverse direction, i.e., from the second reservoir to the first reservoir. In some embodiments the window 302 is about 200 μm thick (e.g., between about 100 and 300 um thick). In some embodiments the spacer 400 is about 0.1 mm to 1 mm. In some embodiments the spacer comprises a pressure sensitive adhesive (PSA) such as a silicone PSA. In some embodiments, cartridge 406 consists of a 50 m thick silicone PSA (400) containing a laser cut channel geometry bonded to a 200 μm thick transparent viewing window (302), wherein the fluidic thru-holes 508 and 510 are 17.5 mm apart, bracketing a 8 mm×8 mm central region inside which spotted microarrays can be imaged. In some embodiments the substrate is silicon and processed using Si processing technology, for example where ports 508 and 510 are through silicon holes that are formed by laser micromachining.

In some embodiments the window 302 is of optical quality, for example made of quartz or glass such as borosilicate glass. In some embodiments the window can be anti-reflection coated on the top surface to minimize the optical reflections. In some embodiments the transparent portion can include a conducting/transparent layer, such as indium tin oxide (ITO).

In some embodiments the substrate 100 is a silicon substrate including a transparent layer, such as silicon oxide or silicon nitride layer. For example, the dimensions can be selected for IRIS or SP-IRIS as taught in International Application (designating the U.S.) nos. PCT/US2006/015566, PCT/US2010/033397, PCT/US2014/062605 and PCT/US2015/019136, which are hereby incorporated by reference, in their entirety.

FIG. 5D is a picture, top down view, of a window and spacer (left) and substrate of a chip, or cartridge, according to some embodiments.

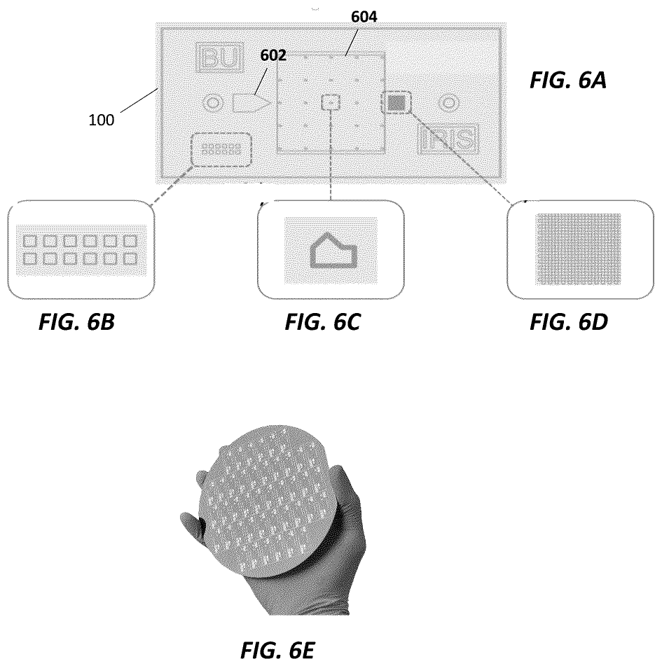

FIG. 6A is a top view of the substrate according to some embodiments. The substrate is configured as a silicon substrate having a top surface transparent layer, such as a dielectric layer of silicon oxide or silicon nitride. The dielectric layer is patterned for several functional purposes such as reflective reference regions, alignment marks for robotic spotting, and autofocusing targets. FIG. 6B is a detailed view showing a chip identification region. FIG. 6C is a detailed view showing a reference region. FIG. 6D is detailed view showing an auto focus region. The pointed arrow-like feature 602 in the center left of the chip forms a high-visibility feature that can be used by template-recognition algorithms in the spotter software to easily locate the channel location for accurate micro-array positioning. Spots are deposited within the central square region 604 measuring, for example 8 mm×8 mm. The two vertical lines bracketing the left and right boundaries of the central region 604 is comprised of a dotted linear array, for example, of 12.5 μm etched squares. These dotted lines are positioned such that they fall within the visible area of the channel, and can be used as a focal reference for initial focal plane identification without requiring the operator to locate specific spot locations. These multiple designed features assist the performance the SP-IRIS.

The central region 604 can include one or more, for example an array or microarray, of functionalized groups. The functional groups can be capture agents, for example, a protein, antibody or complexing agent. Without limitation, this region is configured for capturing at least transiently an analyte for detection, e.g., using SP-IRIS. In some embodiments, for example as shown by the chip design in FIG. 6E, the substrate forms a 25.2 mm by 12.5 mm unit cell. This geometry allows for 42 individual chips to be diced from a 150 mm (6 inch) wafer.

In some embodiments, the fluidic cartridge can be constructed as described by the above figures FIGS. 5A-5D and FIGS. 6A-6E. In this design, holes through the sensor chip facilitates liquid inlet and outlet to the chamber on the surface of the sensor chip. It is desirable to have a chip/cartridge system that can benefit from established Si fabrication processes and achieve low unit cost without investment in infrastructure. In some embodiments using laser micromachining, through holes are drilled on the IRIS chips allowing a much simpler integrated cartridge as shown in the figures. The cartridge is composed of the IRIS chip (with various thickness of oxide or nitride on Si for difference applications) with inlet and outlet holes for fluidics, and a flow channel is formed by attaching a cover glass with a patterned silicon adhesive layer (typically from 0.1 mm to 1 mm thick) to define the channel height.

One of the benefits of this embodiment is that the top window does not require to be machined (patterned or processed). Thus, the top window can have optical and functional properties without incurring significant cost. For example, the cover glass can be anti-reflection coated on one side to reduce the effect of reflections from the air/glass interface, which can significantly affect the image quality in polycarbonate or Cyclic Olefin Copolymer (COC) based windows. For interferometric sensing (IRIS) elimination of undesirable reflections is more significant than other types of optical sensors. The use of standard glass or other high-quality optical windows also provide polarization maintenance that is important for some modalities of IRIS technology, in particular polarization enhanced detection of nanoparticles [Sevenler et al, “Digital Microarrays: Single-Molecule Readout with Interferometric Detection of Plasmonic Nanorod Labels,” ACS Nano, 2018, 12 (6), pp 5880-5887]. Other applications benefiting from the high optical quality include enhanced IRIS with pupil function engineering [Avci et al., “Pupil function engineering for enhanced nanoparticle visibility in wide-field interferometric microscopy,” Optica 2017, 4(2), pp. 247-254]. There are other functional opportunities, for example, a conducting/transparent layer (ITO) can be used on the cover glass to allow for electric field application between the top window and the Si chip. FIG. 6A shows the typical design of the IRIS sensor chip with various identification, alignment and autofocus features as well as a typical implementation on a 150 mm Si wafer size. The chip design (shown in FIG. 6E) forms a 25.2 mm by 12.5 mm unit cell. This geometry allows for 42 individual chips from a 150 mm (6 inch) wafer.

In some embodiments, the chip design can have more than one inlet hole and/or more than one outlet hole for fluidic connections. The cartridge can be constructed to facilitate multiple channels and incubation chambers. The chip dimensions can be larger or smaller depending on the applications and number of channels.

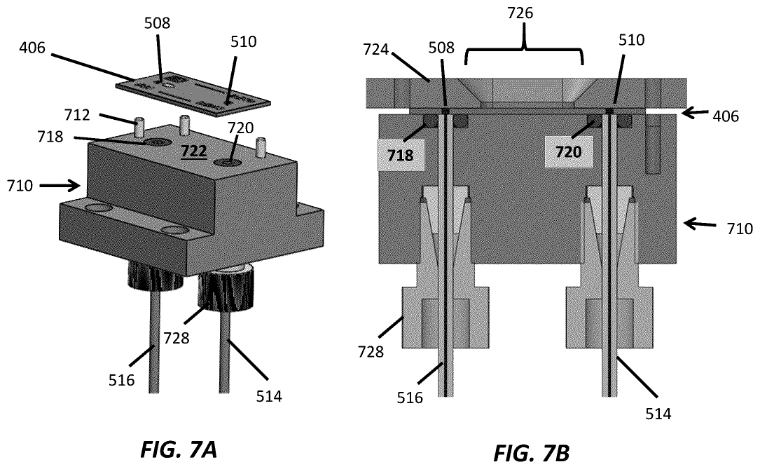

FIGS. 7A, 7B, 8A, 8B, 8C, 9A, 9B, 9C, and 9D show various views and components of a holder to connect the cartridge to a fluidic system. The various embodiments can optionally incorporate the following general features. In some embodiments, the connections between the cartridge or chip substrate and the fluidic manifold include a sealing element such as O-ring seals. In some embodiments, the clamping fixture provides enough clamping force to fully compress these seals, so that the back of the chip substrate is co-planar with the top surface of the manifold. This co-planarity is helps achieve repeatable alignment between chip surface and the optical system. In some embodiments the clamping fixture is easy to assemble as part of the chip mounting process. In some embodiments the clamping mechanism has at least two states—a loading state which allows for easy access to the chip area, and a clamping state in which force is applied to the top of the disposable cartridge. In some embodiment the fixture should be at a local minimum in both of these states (e.g. the user should not have to manually hold open the mechanism during loading). In some embodiments the mechanism applies force evenly across the chip surface as a resulting of a single action from the user (e.g. the user should not be required to coordinate motion between two different components). In some embodiments, the fixture can incorporate a tip/tilt mechanism to adjust angular alignment.

FIG. 7A is an exploded view showing a fluid manifold and cartridge 406. The fluid manifold includes a base component 710 and alignment elements 712 fixed/attached to the base. The base also includes holes configured as conduits. Each conduit can include a hole with a tube placed therethrough, for example, shown as extending out from the based as fluid conduit 516 and fluid conduit 514. The holes can include a sealing element 718 and sealing element 720, for example O-rings. The holes can also include fittings 728 In operation, the cartridge and base are brought together so that a surface of the cartridge opposite the detection region and the base surface 722 are brought together. The alignment pins are positioned on the base for alignment of fluid conduit 516 with the port 508 and for alignment of fluid conduit 514 with port 510, when two edges of the cartridge are contacted with all three alignment pins. For example, one edge of the cartridge contacts a first alignment pin and a second edge of the cartridge contacts a second and third alignment pin.

FIG. 7B shows a front cross section view of a holder and cartridge wherein the holder is shown including a clamping bar 724 as well as the elements shown in FIG. 7A. The clamping bar has an aperture indicated by bracketed region 726 that, when the cartridge is aligned on the base, is in alignment with at least a portion of the transparent portion of the cartridge window.

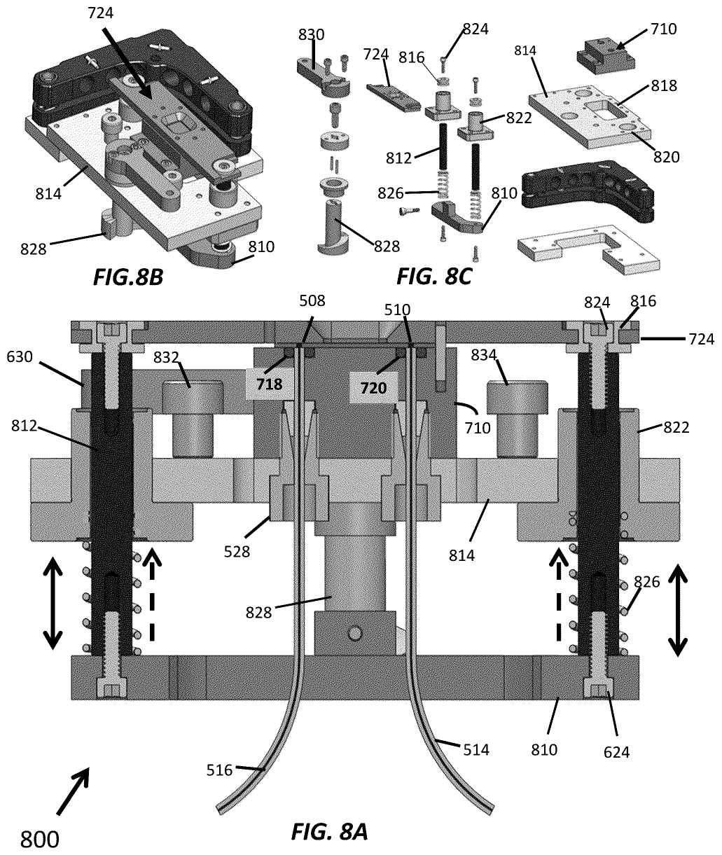

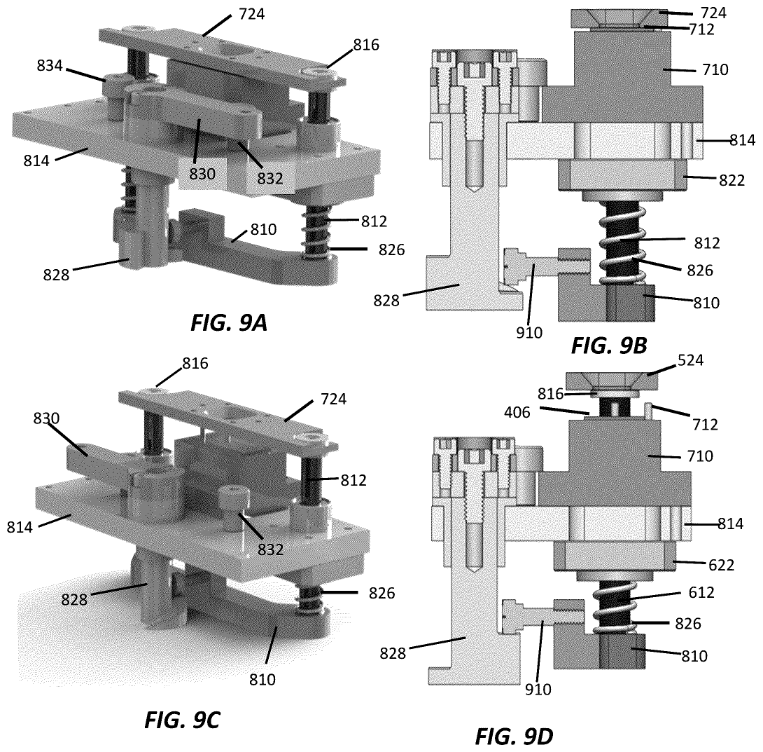

FIG. 8A shows a cross section view of a holder 800 or clamping fixture for cartridges. FIG. 8B is a detail view of some of the components of the holder while FIG. 8C shows details of some components is an exploded view. In addition to the components previously described, the holder includes a bottom platform 810, a guide rails 812, base platform 814, and guiding elements 816. The base platform 814 is configured for holding the base 710, for example base platform 814 can include holes 818 and the base 710 can have corresponding holes that can be used with a fastener, for example a bolt, to attach the base to the platform. The platform 814 can also include holes 820 for inserting guiding elements 812 therethrough. Optionally bearing elements 822 are place in the holes. The guide rails 812 can be attached to bottom platform 810 at a first end, and to the guiding elements 816 on a second end, for example using fastening elements 824. The guiding elements are configured to removably engage with the clamping bar 724. The guiding elements allow aligned or guided movement of the base platform 814 relative to the clamping bar and bottom platform 810 as indicated by the double headed arrow. A tensioning element 826 such as a spring that is coupled to the bottom platform 810 and base platform 814 (e.g., through bearing element 822) provides a force in the direction as indicated by the single headed dashed arrows. This force is provided to the base and cartridge in the same direction as indicted by the dashed arrows and is met by an equal and opposite force by the clamping bar. Therefore, the tensioning elements 826 provide a force for compressing and holding the cartridge. For example, the force can provide a compressive force against the sealing element 718, causing it to deform and form a fluid tight seal between conduits 516 and port 508, and the force can provide a compressive force against the sealing element 720, causing it to deform and form a fluid tight seal between conduits 514 and port 510. Other elements include a rotary cam 828 and cam handle or protrusion 830 and bumpers 832 and 834 and will be described in detail with reference to FIGS. 9A, 9B, 9C and 9D.

FIGS. 9A, 9B, 9C and 9D show the holder 800 and cartridge 406. FIG. 9A is an isometric view where the holder is in a clamped position, while FIG. 9B shows a cross-cut side view in the clamped position. FIG. 9C shows an isometric view of the holder in a loading position, while FIG. 9D show a cross-cut side view in the loading position. Cam follower 910 is attached to bottom platform 810. Rotary cam 828 is mounted to the base platform 814 and is configured for engaging/coupling the bottom platform 810 through a cam follower 910. Cam handle 830 is attached to the rotary cam and can be used to rotate the cam from a clamped position wherein the handle is in contact with bumper 834, to a loading position (180 degrees) wherein the handle is in contact with bumper 832. In some embodiments the cam cylinder has features such as indentations or dents (not shown) providing a local energy minimum to hold the cam follower in the loading position and configured to allow the user to use both hands to load and align the chip against the force applied by the springs.

In some embodiments, the holder 600 provides a force that provides a tight and reliable fluidic seal to the disposable cartridge. In some embodiments the base 510 consists of an aluminum block with two vertical channels placed in line with the fluidic ports 208 and 210 on the cartridge 106. In some embodiments 1/16″ ID FEP tubes, are inserted into the holder channels until the tube end is nearly co-planar with the top surface of the holder and which form fluid flow conduits 216 and 214. For example, fluid travels from an external pump through the conduits. In some embodiments these tubes are locked in place with flangeless chromatography fittings 528 (e.g., available from Upchurch Scientific) and which interface with the underside of the base via ¼-28 threaded ports. In some embodiments two O-rings, 518 and 520, (0.070″ Thickness, 1/16″ ID× 3/16″ OD) seal against both the back of the cartridge 106 substrate and the protruding tip of the tubes, for example, which both minimizes dead volume and prevents sample fluid from wicking into the gap between the tubes and the manifold. This double seal also eliminates the need to clean the manifold itself between experiments, as sample fluid does not interact with permanent components other than the tubes themselves. The clamping force required for full compression of the O-ring seals is dictated the gland depth and the O-ring durometer. In some embodiments a soft durometer O-ring material (e.g., 50 Shore A silicone) is used to minimize the amount of force required while maintaining an effective seal. Using the minimum recommended compression depth of 20% to ensure reliable seal integrity despite manufacturing tolerances, a gland depth of 0.056″ for a 0.070″ Thick O-ring can be used, and a clamping force of about 7 lbf per seal for full compression.

In some embodiment for holder 800 the design uses compression springs 826 to provide sealing force. This design, as previously described utilizes a rotary cam to control the position of a removable clamping bar. The user can switch between the clamping state and the loading state by rotating the cam handle by 180 degrees, which activates a cam follower connected to the spring carriage. Detents in the cam cylinder “capture” the cam follower in the raised (loading) state, allowing the user to use both hands to load and align the chip despite the significant force applied by the compressed springs. Shoulder bolts mounted adjacent to the handle constrain the motion of the cam to 180 degrees by blocking the motion of the handle, in order to prevent a system malfunction resulting from the user accidentally over-rotating the cam mechanism. A clamping bar interfaces with two slots on the end of the guide rods that constrain the motion of the spring carriage, and can be easily removed and replaced to allow for unhindered access to the fluid manifold for chip mounting.

In addition, the cam holder contains multiple features to assist in the ease of assembly and disassembly of the system for maintenance and reconfiguration purposes. Removing the handle from the cam assembly enables the cam cylinder to rotate more than 180 degrees, where, in some embodiments, a cutout in the cam ramp allows the cam follower to pass through for easy assembly and disassembly purposes.

In some embodiments the following steps can be used for making the silicon substrate. Silicon wafers are acquired from a wafer supplier e.g., Silicon Valley Microelectronics-SVM (California, US). Six-inch silicon wafers with 100 nm thermal oxide grown (SiO2) or 100 nm SiN deposited thereon can be used. A photoresist is applied and a mask for the desired patterning is used (e.g., alignment marks and marks indicating the location of the through holes. After etching the desired pattern, the photoresist is removed and a fresh photoresist coating is applied. Laser machining (Potomac lasers, MD, USA) can be used to drill the liquid holes in the wafers. The chips are cut (sawed) and then the photoresist is removed. After this, processing steps for functionalizing the surface (e.g., silanes, oxygen plasma and then a polymer and then then capture agents such as functional groups, DNA, proteins are attached. A glass cover window with the spacer layer is then adhered to the surface.

It has been found that laser machining is sufficiently clean, and with acceptable topography for embodiments of fluidic cartridges and affords efficient formation of holes though. Other methods for making holes are not as satisfactory because the holes need to be several hundred microns thick. For example, 4″ wafers are 400-500 microns thick. Reactive ion etching can be used but it is expensive with multiple steps/layers/repetitions required to form holes through the wafer. Larger Si wafers are typically thicker, for example, 6″ wafers are about 625 microns thick. Another method, anisotropic etching e.g., using KOH, provides clean conical holes but is only practical for a couple hundred microns thick wafers.

Embodiments of the various aspects described herein can be illustrated by the following numbered paragraphs.
1. An optical biosensing cartridge comprising:
   a substrate with through holes as ports to facilitate liquid flow,
   a cover window having a transparent portion,
   a spacer separating the cover window from the substrate by a predefined distance,
   a channel extending from one port to a different port, the channel defined by the substrate, spacer and cover film, and
   a detection region on the substrate at least partially in the channel, wherein the detection region includes at least one dielectric layer having a predefined uniform thickness.
2. The optical biosensing cartridge according to paragraph 1, wherein the substrate comprises silicon.
3. The optical biosensing cartridge according to paragraph 1 or 2, wherein the detection region includes a layer of silicon oxide on a silicon substrate.
4. The optical biosensing cartridge according to any one of paragraphs 1-3, wherein the detection region includes a layer of silicon nitride on a silicon substrate.
5. The optical biosensing cartridge according to any one of paragraphs 1-4, wherein the through holes are formed by laser micromachining.
6. The optical biosensing cartridge according to any one of paragraphs 1-5, wherein there the through holes include one through hole for liquid inlet and one through hole for liquid outlet.
7. The optical biosensing cartridge according to any one of paragraphs 1-6, wherein the spacer comprises an adhesive.
8. The optical biosensing cartridge according to any one of paragraphs 1-7, wherein the cover window is glass and includes an anti-reflection coating on a top surface.
9. The optical biosensing cartridge according to any one of paragraphs 1-8, wherein the cover window comprises an optical grade transparent material such as quartz or borosilicate glass.
10. The optical biosensing cartridge according to any one of paragraphs 1-9, wherein the detection region includes one or more of identification regions, alignment marks for robotic spotting, reflective reference regions and autofocus regions.
11. The optical biosensing cartridge according to any one of paragraphs 1-10, wherein the substrate is a silicon chip, and wherein the ports comprise a first port and the second port is configured as through the Si chip holes manufactured by laser micromachining, the spacer includes a pressure sensitive adhesive, the detection area includes a dielectric layer of silicon oxide or silicon nitride between 50-200 nm thick, and the cover window comprises silicate glass having one surface comprising an anti-reflective coating.
12. An apparatus comprising:
   the optical biosensing cartridge according to any one of paragraphs 1-11 and a holder;
   wherein the holes of the cartridge include a first port and a second port and the channel extends between the first port and the second port,
   wherein the holder includes,
      a base having a first fluid conduit coupled to a first port, and a second fluid conduit coupled to a second port,
      a clamping element for removably fastening the cartridge to the base;
wherein the first conduit, the first port, the channel, the second port and the second conduit define a fluid flow path from the first fluid conduit to the second fluid conduit.
13. An apparatus comprising:
   a removable cartridge and a holder;
   wherein the cartridge includes,
      a substrate having a first port and a second port,
      a cover film having a transparent portion,
      a spacer separating the cover film from the substrate by a predefined distance,
      a channel extending from the first port to the second port, the channeled defined by the substrate, spacer and cover film, and
      a detection region on the substrate at least partially in the channel, wherein the detection region includes one or multiple dielectric layers having a predefined uniform thickness;
      wherein the holder includes,
      a base having a first fluid conduit coupled to the first port, and a second fluid conduit coupled to the second port,
      a clamping element for removably fastening the cartridge to the base;
   wherein the first conduit, the first port, the channel, the second port and the second conduit define a fluid flow path from the first fluid conduit to the second fluid conduit.
14. The apparatus of paragraph 13, wherein the base includes an alignment element including at least three pins positioned to alignment the cartridge with the base whereby the first fluid conduit is aligned with first port of the cartridge and the second fluid conduit is aligned with the second port of the cartridge.
15. The apparatus according to any one of paragraphs 13-14, wherein the transparent portion comprises an optical grade transparent material such as quartz or borosilicate glass.
16. The apparatus according to any one of paragraphs 13-15, wherein the transparent portion is aligned with the detection region.
17. The apparatus according to any one of paragraphs 13-16, wherein the detection region includes one or more of identification regions, alignment marks for robotic spotting, reflective reference regions and autofocus regions.
18. The apparatus according to any one of paragraphs 13-17, wherein the clamping element includes:
   a platform supporting the base,
   a rotary cam mounted to the platform and engaging a bottom platform through a cam follower mounted on the bottom platform, wherein the rotary cam has a loading position and a clamped position,
   a clamping bar for engaging the cartridge and compressing the cartridge against the base, the clamping bar having an aperture in alignment with at least a portion of the transparent portion,
   at least one guide rail mechanically coupled to the platform and bottom platform and for holding the platform and clamping bar in alignment during operation of the rotary cam during a clamping operation,
   at least one spring mechanically coupled to the base, bottom platform, clamping bar and the cam follower,
   wherein when the rotary cam is in the clamped position the bar provides a compressive force through the spring against the cartridge, and when the rotary cam is in a loading position the cartridge can be disengaged and removed from the base.
19. The apparatus according to any one of paragraphs 13-18, further comprising a first sealing element disposed on the base at one end of the first fluid conduit for sealing a connection between the first fluid conduit and the first port, and a second sealing element disposed on the base at one end of the second fluid conduit for sealing a connection between the second fluid conduit and the second port.
20. The apparatus according to paragraph 19, wherein the rotary cam includes a handle and the platform includes a first bumper and a second bumper for engaging the handle and restricting the rotational motion of the cam to the clamped and unclamped position.
21. A system for measuring particles comprising;
   the apparatus of any one of paragraphs 13-20,
   an interferometric reflectance imaging sensor (IRIS) system comprising an objective lens for illuminating the detection region of the cartridge and collecting reflected light from the detection region,
   and
   a stage for holding the apparatus and for moving the apparatus relative to the objective lens.
22. The system of paragraph 21 wherein the substrate is a first reflective surface, the dielectric layer comprises a second reflective surface, and single particles are detected on the dielectric surface by the IRIS system.
23. A method of measuring particles comprising,
   flowing an analyte solution including particles through the channel and the detection region of the apparatus of any one of paragraphs 13-20
   measuring the particles in the detection region using the system of paragraphs 21 or 22.
24. A method of measuring biological mass accumulating on the sensor surface,
   flowing an analyte solution including biomolecules through the channel and the detection region of the apparatus of any one of paragraphs 13-20
   measuring the surface accumulation of biomolecules in the detection region using the system of paragraph 21 or 22.

Other embodiments are within the scope and spirit of the invention. Further, while the description above refers to the invention, the description may include more than one invention.

What is claimed is:

1. An optical biosensing cartridge comprising:
a substrate with through holes as ports to facilitate liquid flow,
a cover window having a transparent portion,
a spacer separating the cover window from the substrate by a predefined distance,
a channel extending from one port to a different port, the channel defined by the substrate, spacer and cover film, and
a detection region on the substrate at least partially in the channel, wherein the detection region includes at least one dielectric layer having a predefined uniform thickness.

2. The optical biosensing cartridge according to claim 1, wherein the substrate comprises silicon.

3. The optical biosensing cartridge according to claim 1, wherein the detection region includes a layer of silicon oxide on a silicon substrate.

4. The optical biosensing cartridge according to claim 1, wherein the detection region includes a layer of silicon nitride on a silicon substrate.

5. The optical biosensing cartridge according to claim 1, wherein the through holes are formed by laser micromachining.

6. The optical biosensing cartridge according to any one of claim 1, wherein there the through holes include one through hole for liquid inlet and one through hole for liquid outlet.

7. The optical biosensing cartridge according to any one of claim 1, wherein the spacer comprises an adhesive.

8. The optical biosensing cartridge according to claim 1, wherein the cover window is glass and includes an anti-reflection coating on a top surface.

9. The optical biosensing cartridge according to claim 1, wherein the cover window comprises an optical grade transparent material such as quartz or borosilicate glass.

10. The optical biosensing cartridge according to claim 1, wherein the detection region includes one or more of identification regions, alignment marks for robotic spotting, reflective reference regions and autofocus regions.

11. The optical biosensing cartridge according claim 1, wherein the substrate is a silicon chip, and wherein the ports comprise a first port and the second port is configured as through the Si chip holes manufactured by laser micromachining, the spacer includes a pressure sensitive adhesive, the detection area includes a dielectric layer of silicon oxide or silicon nitride between 50-200 nm thick, and the cover window comprises silicate glass having one surface comprising an anti-reflective coating.

12. An apparatus comprising:
the optical biosensing cartridge according to any one of claim 1 and a holder;
wherein the holes of the cartridge include a first port and a second port and the channel extends between the first port and the second port,
wherein the holder includes,
a base having a first fluid conduit coupled to a first port, and a second fluid conduit coupled to a second port,
a clamping element for removably fastening the cartridge to the base;
wherein the first conduit, the first port, the channel, the second port and the second conduit define a fluid flow path from the first fluid conduit to the second fluid conduit.

13. An apparatus comprising:
a removable cartridge and a holder;
wherein the cartridge includes,
a substrate having a first port and a second port,
a cover film having a transparent portion,
a spacer separating the cover film from the substrate by a predefined distance,
a channel extending from the first port to the second port, the channeled defined by the substrate, spacer and cover film, and
a detection region on the substrate at least partially in the channel, wherein the detection region includes one or multiple dielectric layers having a predefined uniform thickness;
wherein the holder includes,
a base having a first fluid conduit coupled to the first port, and a second fluid conduit coupled to the second port,
a clamping element for removably fastening the cartridge to the base;
wherein the first conduit, the first port, the channel, the second port and the second conduit define a fluid flow path from the first fluid conduit to the second fluid conduit.

14. The apparatus of claim 13, wherein the base includes an alignment element including at least three pins positioned to alignment the cartridge with the base whereby the first fluid conduit is aligned with first port of the cartridge and the second fluid conduit is aligned with the second port of the cartridge.

15. The apparatus according to claim 13, wherein the transparent portion comprises an optical grade transparent material such as quartz or borosilicate glass.

16. The apparatus according to claim 13, wherein the transparent portion is aligned with the detection region.

17. The apparatus according to any claim 13, wherein the detection region includes one or more of identification regions, alignment marks for robotic spotting, reflective reference regions and autofocus regions.

18. The apparatus according to claim 13, wherein the clamping element includes:
a platform supporting the base,
a rotary cam mounted to the platform and engaging a bottom platform through a cam follower mounted on the bottom platform, wherein the rotary cam has a loading position and a clamped position,
a clamping bar for engaging the cartridge and compressing the cartridge against the base, the clamping bar having an aperture in alignment with at least a portion of the transparent portion,
at least one guide rail mechanically coupled to the platform and bottom platform and for holding the platform and clamping bar in alignment during operation of the rotary cam during a clamping operation,
at least one spring mechanically coupled to the base, bottom platform, clamping bar and the cam follower,
wherein when the rotary cam is in the clamped position the bar provides a compressive force through the spring against the cartridge, and when the rotary cam is in a loading position the cartridge can be disengaged and removed from the base.

19. The apparatus according of claim 18, further comprising a first sealing element disposed on the base at one end of the first fluid conduit for sealing a connection between the first fluid conduit and the first port, and a second sealing element disposed on the base at one end of the second fluid conduit for sealing a connection between the second fluid conduit and the second port.

20. The apparatus according to claim 19, wherein the rotary cam includes a handle and the platform includes a first bumper and a second bumper for engaging the handle and restricting the rotational motion of the cam to the clamped and unclamped position.
